(19) United States
(12) Patent Application Publication
(10) Pub. No.: US 2020/0095614 A1
(43) Pub. Date: Mar. 26, 2020

(54) METHOD FOR INCREASING THE PRODUCTION OF ETHANOL FROM CORN FIBER IN A STARCH HYDROLYSIS PROCESS

(71) Applicant: DANISCO US INC., Palo Alto, CA (US)

(72) Inventors: Jeffrey V. Miller, Palo Alto, CA (US); Bradley Roger Kelemen, Palo Alto, CA (US); Pim Van Der Kley, Palo Alto, CA (US); Igor Nikolaev, Palo Alto, CA (US); Su Yin Marina Chow, Palo Alto, CA (US); Monica Tse, Palo Alto, CA (US); Jing Ge, Palo Alto, CA (US); Zhongmei Tang, Palo Alto, CA (US); Kirstin Yasuko Nose Crotty, Palo Alto, CA (US)

(21) Appl. No.: 16/469,721

(22) PCT Filed: Dec. 15, 2017
(86) PCT No.: PCT/US17/66737

§ 371 Date: Jun. 14, 2019

Related U.S. Application Data
(60) Provisional application No. 62/434,883, filed on Dec. 15, 2016.

(51) Int. Cl. C12P 7/14; C12P 19/02; C12N 9/24

(52) U.S. Cl. CPC C12P 7/14; B01D 3/002; C12N 9/2402; C12P 19/02

(57) ABSTRACT

Described are compositions and methods relating the use of α-glucosidase to increase the production of ethanol from a corn fiber product in a starch hydrolysis process.

### CROSS-REFERENCE TO RELATED APPLICATIONS

This application claims the benefit of U.S. Provisional Application No. 62/434,883, filed Dec. 15, 2016, the disclosure of which is incorporated herein by reference in its entirety.

### TECHNICAL FIELD

The present compositions and methods relate to the use of α-glucosidase to increase the production of ethanol from a corn fiber material derived from a predominantly corn kernel feedstock.

### BACKGROUND

Many commercial fermentation process are designed to maximize the conversion of plant-derived complex carbohydrates into simple fermentable sugars. These sugars may be simultaneously or sequentially utilized by microorganisms to produce commercially-valuable biochemicals or biofuels. One well-known such process is the conversion of starch into glucose followed by yeast fermentation to produce ethanol. A commonly used feedstock is corn kernels, which may include some corn stover, resulting in a feedstock that is predominantly starch but contains a significant amount of corn fiber.

Corn fiber is sometimes separated from corn carbohydrates by mechanical fractionation, resulting in essentially two (or more) different feedstocks having different chemical and enzymatic processing requirements. In addition, corn-ethanol processing generates by-products that include non-fermentable plant materials. There is considerable interest in treating corn fiber and corn processing by-products that contain corn fiber to extract more fermentable sugars, thereby improving the overall yield of commercially valuable biochemicals or biofuels.

### SUMMARY

The present compositions and methods relate to the use of α-glucosidase to increase the production of ethanol from a corn fiber product in a starch hydrolysis process. Aspects and embodiments of the compositions and methods are described in the following, independently-numbered paragraphs.

1. In one aspect, a method for increasing the production of fermentable sugars in an ethanol fermentation process is provided, comprising: chemically-pretreating corn fiber material derived from a predominantly corn kernel feedstock; and contacting the pretreated corn fiber with α-glucosidase to release fermentable sugars from the corn fiber; wherein the contacting with α-glucosidase releases fermentable sugars from the corn fiber material derived from a predominantly corn kernel feedstock that otherwise would not be available for fermentation.

2. In some embodiments of the method of paragraph 1, the corn fiber product is the stillage resulting from distillation of a fermentation product, or a derivative, thereof

3. In some embodiments of the method of paragraph 1, the corn fiber product is produced by physically separating corn fiber from starch in a substrate for ethanol fermentation.

4. In some embodiments of the method of paragraph 1, the corn fiber product is exposed to a temperature of at least 80° C. prior to the contacting with α-glucosidase.

5. In some embodiments of the method of paragraph 1, the corn fiber product is subjected to an alcohol distillation step prior to the contacting with α-glucosidase.

6. In some embodiments of the method of paragraph 1, the corn fiber product contains between about 1-12% corn fiber (wt/wt).

7. In some embodiments of the method of any of the preceding paragraphs, the pretreatment is acid pretreatment.

8. In some embodiments of the method of any of the preceding paragraphs, the pretreatment is alkaline pretreatment.

9. In some embodiments of the method of any of the preceding paragraphs, the α-glucosidase is a GH31 enzyme.

10. In some embodiments of the method of any of the preceding paragraphs, the α-glucosidase has at least 70% amino acid sequence identity to a polypeptide having an amino acid sequence selected from the group consisting of SEQ ID NO: 1, SEQ ID NO: 2, SEQ ID NO: 3 and SEQ ID NO: 4.

11. In some embodiments of the method of any of the preceding paragraphs, the contacting with α-glucosidase is performed in the presence of one or more additional enzymes.

12. In another aspect, a method for increasing the production of fermentable sugars from chemically-pretreated stillage is provided, comprising: fermenting a predominantly corn kernel feedstock with an ethanolagen to produce ethanol; evaporating at least a portion of the ethanol and recovering stillage; chemically-pretreating the stillage; contacting the stillage with α-glucosidase to release fermentable sugars; fermenting the released fermentable sugars with an ethanolagen to produce additional ethanol; and recovering the additional ethanol; wherein the contacting with α-glucosidase releases fermentable sugars from the corn fiber material derived from the predominantly corn kernel feedstock that otherwise would not be available for fermentation.

13. In some embodiments of the method of paragraph 12, the pretreatment is acid pretreatment.

14. In some embodiments of the method of paragraph 12, the pretreatment is alkaline pretreatment.

15. In some embodiments of the method of any of paragraphs 12-14, the corn fiber product contains between about 1-12% corn fiber (wt/wt).

16. In some embodiments of the method of any of paragraphs 12-15, the α-glucosidase is a GH31 enzyme.

17. In some embodiments of the method of any of paragraphs 12-16, the α-glucosidase has at least 70% amino acid sequence identity to a polypeptide having an amino acid sequence selected from the group consisting of SEQ ID NO: 1, SEQ ID NO: 2, SEQ ID NO: 3 and SEQ ID NO: 4.

18. In some embodiments of the method of any of paragraphs 12-17, the contacting with α-glucosidase is performed in the presence of one or more additional enzymes.

19. In another aspect, a polypeptide having α-glucosidase activity is provided, selected from the group consisting of:

(a) a polypeptide comprising an amino acid sequence having preferably at least 94% identity to the polypeptide of SEQ ID NO: 3;

(b) a polypeptide encoded by a polynucleotide that hybridizes under preferably at least low stringency conditions, more preferably at least medium stringency conditions, even more preferably at least medium-high stringency conditions, most preferably at least high stringency, and even most preferably at least very high stringency conditions with
(i) the mature polypeptide coding sequence of SEQ ID NO: 7,
(ii) the genomic DNA sequence comprising the mature polypeptide coding sequence of SEQ ID NO: 7, or
(iii) a full-length complementary strand of (i) or (ii);

(c) a polypeptide encoded by a polynucleotide comprising a nucleotide sequence having preferably at least 94% identity to the polypeptide coding sequence of SEQ ID NO: 3;

(d) a variant comprising a substitution, deletion, and/or insertion of one or more (e.g., several) amino acids of the polypeptide of SEQ ID NO: 3;

(e) a mature polypeptide produced by the processing of the polypeptide of SEQ ID NO: 6 by a signal peptidase or post translational modification during secretion from an expression host; and

(f) a fragment of a polypeptide of (a), (b), (c), (d), or (e) that has α-glucosidase activity.

20. In another aspect, a polynucleotide comprising a nucleotide sequence that encodes the polypeptide of paragraph 19 is provided.

These and other aspects and embodiments of present compositions and methods will be apparent from the description, including the accompanying Figures.

### BRIEF DESCRIPTION OF THE DRAWINGS

FIG. 1 is a plasmid map of pTTT-PvaGlu1.

FIG. 2 is a graph showing the amount of glucose (mg/mL) released from a whole stillage substrate using various amounts of different α-glucosidases (μg/g dry solids) at pH 5 and 50° C. for 24 hours.

### DETAILED DESCRIPTION

### I. Introduction

The present compositions and methods relate to an improvement on current methods that utilize enzymes to release fermentable sugars from plant-derived carbohydrates. Specifically, the compositions and methods involve the addition of α-glucosidase, which has a complex substrate specificity, to corn fiber derived from a predominantly corn kernel feedstock that has been pretreated in preparation for enzymatic digestion. The present compositions and methods should not be confused with the addition of α-glucosidase to a cellulosic feedstock in a cellulose-based biofuel process.

### II. Definitions

Prior to describing the present compositions and methods in detail, the following terms are defined for clarity. Terms not defined should be accorded their ordinary meanings as used in the relevant art.

As used herein, an “α-glucosidase” (EC 3.2.1.20) is an enzyme capable of the hydrolysis of terminal, non-reducing, a (1-4)-linked-D-glucose residues from a substrate, with the resulting release of D-glucose. α-glucosidases are not to be confused with β-glucosidases (E.C. 3.2.1.21) or other carbohydrate-processing enzymes, including but not limited to α-amylases (EC 3.2.1.1), β-amylases (EC 3.2.1.2), glucoamylases (EC 3.2.1.3), trehalases (EC 3.2.1.28), pullulanses (EC 3.2.1.41), and other enzymes having predominantly different substrate specificities.

As used herein, “background α-glucosidase activity,” and similar terms, refer to enzyme activity that is not attributable to the addition of exogenous α-glucosidase to plant fiber.

As used herein, “physical treatment,” or similar expressions, refer to mechanical and/or thermal treatments, which may or may not result in chemical modifications, that modifying a fiber substrate to increase its suitability for digestion by enzymes, particularly α-glucosidase.

As used herein, “thermal pretreatment” refers to exposure to a temperature suitable to evaporate alcohol, particularly ethanol, as in the case of recovering fuel alcohol from a fermentation process, or to otherwise modify a fermentation substrate by heat, so as to modify the substrate to increase enzymatic digestion. Thermal pretreatment typically involves a temperature of at least 80° C.

As used herein, “chemical treatment” refers to non-enzymatic modification of a substrate, such as by acid or alkaline treatment (i.e., pH adjustment), exposure to oxidizing or reducing agents, and the like.

As used herein, a “predominantly corn kernel feedstock” is a feedstock containing at least 80%, at least 85%, at least 90%, at least 95%, at least 97% and even at least 98% corn kernels. The remaining material may include corn stover, other plant components, insects and the like.

As used herein, a “corn fiber product” is material produced as a result of processing a predominantly corn kernel-based substrate in a typical fuel ethanol processing plant, where the steps of starch hydrolysis and fermentation are performed to produce ethanol. An exemplary corn fiber product is whole stillage.

As used herein, “wet distillers' grains (WDG)” primarily contain unfermented grain residues, specifically protein, fiber, fat and up to 70% moisture.

As used herein, “dried distillers' grains with solubles (DDGS) is WDG that has been dried with the concentrated thin stillage (liquid remaining after boiling of the ethanol) to about 10-12 percent moisture. The fiber content can be up to about 5-50%.

As used herein, the singular articles “a,” “an,” and “the” encompass the plural referents unless the context clearly dictates otherwise. All references cited herein are hereby incorporated by reference in their entirety. The following abbreviations/acronyms have the following meanings unless otherwise specified:

EC enzyme commission

CAZy carbohydrate active enzyme

w/v weight/volume

w/w weight/weight

v/v volume/volume

wt % weight percent

° C. degrees Centigrade

g or gm gram

microgram

mg milligram

kg kilogram

μL and μl microliter

mL and ml milliliter

mm millimeter

μm micrometer

mol mole

mmol millimole

M molar

mM millimolar

μM micromolar

nm nanometer

U unit

ppm parts per million

hr and h hour

EtOH ethanol

III. α-glucosidases

All embodiments of the present compositions and methods relate to the use of α-glucosidase to improving ethanol production from corn fiber derived from a predominantly corn kernel feedstock.

α-glucosidases (EC 3.2.1.20) hydrolyze terminal, non-reducing α-1,4-linked glucose residues in various substrates, releasing glucose. They degrade disaccharides and oligosaccharides quickly while polysaccharides are attacked more slowly. α-glucosidases display broad substrate specificity for α-glucosides, which include panose, isomaltose, isopanose, maltotriose, turanose, and maltose. Aside from being able to hydrolyze synthetic α-glucoside and oligosaccharide substrates, α-glucosidases also can hydrolyze α-glucans such as soluble starch and glycogen.

α-glucosidases can be divided into three types based on substrate specificity. Type I α-glucosidases hydrolyze heterogeneous substrates, such as aryl glucosides and sucrose, more efficiently than they hydrolyze maltose. Type II α-glucosidases prefer maltose and isomaltose as substrates, and have low activities toward aryl glucosides. Type III α-glucosdiases have the same substrate specificity as type II but can additionally hydrolyze polysaccharides such as amylose and starch.

α-glucosidase activity can be found in a wide variety of organisms. α-glucosidases from bacteria, yeast, and insects typically belong to the GH13 class, and have four conserved sequence regions. α-glucosidases from plants, animals, molds, and two species of bacteria are members of GH31 class. Additional α-glucosidase of members of GH97 class.

In some embodiments of the present compositions and methods, the α-glucosidase are of the GH31 class, as described in, e.g., Okuyama, M. et al. (2016) Cell. Mol. Life Sci. 73:2727-51. Exemplary GH31 enzymes are E1, E2, E3 and E4, having the amino acid sequences of SEQ ID NO: 1, SEQ ID NO: 2, SEQ ID NO: 3 and SEQ ID NO: 4, respectively, as shown, below:

E1 GH31 α-glucosidase (SEQ ID NO: 1):
STTAPSQPQFTIPASADVGAQLIANIDDPQAADAQSVCPGYKASKVQHNSRGFTASLQLAGRP
CNVYGTDVESLTLSVEYQDSDRLNIQILPTHVDSTNASWYFLSENLVPRPKASLNASVSQSDL
FVSWSNEPSFNFKVIRKATGDALFSTEGTVLVYENQFIEFVTALPEEYNLYGLGEHITQFRLQ
RNANLTIYPSDDGTPIDQNLYGQHPFYLDTRYYKGDRQNGSYIPVKSSEADASQDYISLSHGV
FLRNSHGLEILLRSQKLIWRTLGGGIDLTFYSGPAPADVTRQYLTSTVGLPAMQQYNTLGFHQ
CRWGYNNWSDLADVVANFEKFEIPLEYIWTDIDYMHGYRNFDNDQHRFSYSEGDEFLSKLHES
GRYYVPIVDAALYIPNPENASDAYATYDRGAADDVFLKNPDGSLYIGAVWPGYTVFPDWHHPK
AVDFWANELVIWSKKVAFDGVWYDMSEVSSFCVGSCGTGNLTLNPAHPSFLLPGEPGDIIYDY
PEAFNITNATEAASASAGASSQAAATATTTSTSVSYLRTTPTPGVRNVEHPPYVINHDQEGHD
LSVHAVSPNATHVDGVEEYDVHGLYGHQGLNATYQGLLEVWSHKRRPFIIGRSTFAGSGKWAG
HWGGDNYSKWWSMYYSISQALSFSLFGIPMFGADTCGFNGNSDEELCNRWMQLSAFFPFYRNH
NELSTIPQEPYRWASVIEATKSAMRIRYAILPYFYTLFDLAHTTGSTVMRALSWEFPNDPTLA
AVETQFMVGPAIMVVPVLEPLVNTVKGVFPGVGHGEVWYDWYTQAAVDAKPGVNTTISAPLGH
IPVYVRGGNILPMQEPALTTREARQTPWALLAALGSNGTASGQLYLDDGESIYPNATLHVDFT
ASRSSLRSSAQGRWKERNPLANVTVLGVNKEPSAVTLNGQAVFPGSVTYNSTSQVLFVGGLQN
LTKGGAWAENWVLEW
E2 GH31 α-glucosidase (SEQ ID NO: 2):
AAIVRRNGASPSCPGYKASNVKTVDGEIVSADLNLAGPACNVYGTDLDDLKLQVEYQSEQRLH
VKIYDAAEQVYQVPTAVLPRPSSANIPPAKSDLKFSMTNDPFSFTIKRRSNGEILFDTSGHPL
IFESQYLGLRTKLPDSPNIYGLGEHTGSFRLPTKNYTRTLWSRDAYGTPKDTNLYGNHPVYFD
YRGSNGTHGVFLLNSNGMDVDIDVDSDGQYLQYNTLGGVLDFYFLSGPDPKAVATQYAETVGK
PVMMPYWGFGFHNCRYGYQDIYEVAEIIANYSAANIPLETQWTDIDYMDLRKVFTLDPYRYPL
KLVQEVVSYLHKHNQHYIMMVDPAVAYQNYSAFNNGVAADAFLKFSNGSIYQGVVWPGPTAFP
DWFAPQTQEFWNSEFSTFFDPAHGVDIDALWIDMNEASNFCDFPCSNPAAYAAANGDPPTPPP
VRLSPPRPIPGFGPDFQPTCVATVSFDCDAQTYFGENILILGNSTTLGAGDVHMAPVMSANNY
PIWQLTVQMPPNGTFSYQYVRKESDGSYIYEQTNRTVTTGDCTSGTLKVSDTITTSSGPHKRS
ELRPLVRSPFPAEDLTRRQSGSMLGLPNRNLLNPPYTIHNAAGNLSEKTINTDLIHAGGYAEY
DTHNLYGTMMSATSREAMLNRRPAVRPLVITRSTFAGAGRQVGHWLGDNFADWDHYRWTIAEL
QEFAALFQIPMVGSDICGYDGNTTDNLCSRWVFLGAFSPFFRDHSDNQSPPHELYRTPQIAAA
ARAAIDIRYRLLDYAYTVLWTQTQTGAPMLNPMFFEYPADSNTADLQYQFFWGDSIMVAPVTD
NDSTTVNVYFPKDQFYDFYTGAPVSGEGNTVTLTDVGFDTIPLYFKGGSIVPMRVRSANTTAE
LRQQDFVVVIAPDSHGDATGQLYLDDGESINQPHTSEIQFSYRGGHFSMTGKFDYDPGNVVIS
QITLLGADGAGKGGSYNSTTKVATYKVNAKLTGKFEASLH
E3 GH31 α-glucosidase (SEQ ID NO: 3), also referred to, herein,
as PvaGlu1:
AAISPAATSSTASWGPVFTVPASADEGAQLTANINDPQSVNAQTVCPGYVASNVQNNEFGFTA
TLNLAGKACNVYGTDVDSLNLTVQYQASDRLNINIGPAHVDASNESWYILSDDLVYKPTVDGT
ASISQSDLLVSWSNEPSFNFKVIRKANKDVLFNTEGTVLVYENQFIEFVSALPENYNLYGLGE
RIHGLRLGNNFTATTYAADAADPIDANTYGTHPFYLDTRYYEVDSKQGTYTLLTTNETDQSKE
YTSFSHGVFLRNAHGQEVLLRPEGITWRTLGGSIDLYFYSGPTQADVTRSYQTSTVGLPTMQQ
YYTFGYHQCRWGYQNWSVMADVVSSFAKFQIPLETIWSDIDYMNAYRDFENDPIRFSYSEGAE
FLGQLHENGQHWVPIVDSAIYIPNSENASDAYDVYTRGEADGVWMTNPDGSLYIGAVWPGYTV
FPDWHNPKAHEFWSNEIATWHQKVAFDGIWIDMSEVSSFCVGSCGTGNLTLNPVHPSFLLPGE
PGAVIYDYPESFNVTNSTEAASASAASVSQAAATASASASTTTSYLRTTPTPGVRDVNHPPYV
INNVQPGHDLAVHAVSPNATHIDGVSEYDVHNLWGYQILNATYHGLLKVWEDKRPFIIGRSTF
AGSGKWAGHWGGDNTSLWAYMFFSIPQALSFSLFGIPMFGVDTCGFNGNSDEELCNRWMQLSA
FFPFYRNHNVLSAISQEPYVWASVIDASKAAMKIRYALLPYIYTLFYLAHTTGSTVMRAVSWE
FPNDPSLAAIDTQFLLGPSLMVVPVLEPQVDYVKGVFPGVGNGEVWYDWYTQSVFDAKPGVNT
TISAPLGHIPVFVRGGSILPMQEPALTTRDARKTPWALLTALGGNGTASGQLYIDDGESITPN
ATLNVDFVASNSNLVASPRGSWVEKNPLANVTVLGVPTAPSSVTFNGAAVPRASVAYNSTSKT
LFVGGLQDFTKTGAWADKWVLKW
E4 GH31 α-glucosidase (SEQ ID NO: 4):
AATQTSSSAYVQTTLESSVDVGANLIANIDDPEATNAQSACPGYRASNVQNTSRGWTATLKLA
GKACNVYGTDVESLNFTLEYLSSTRVNIQITPSHVDSSNASWYHLSEDVVPRPKADKNASAKD
SHFEVSWSNEPSFGFKVARKATGDVLFNTIGSKLVYENQFIEFVTALPDDYNLYGLGEHIQQL
RLLKNSTFTLYAADTGDPVDLNTYGSHAFYLDTRYYEVNDKGSHTLVSSDQATTSKNYVSYSH
GVFLRNAHGQEILLGTGKLTWRTIGGSIDLTLYAGPTQTEVTKDYQLSTIGLPAMQQYFTFGY
HQCRWGYTNWSEVEDVVANFQKFEIPLENIWNDIDYMHGYRDFDNDQNRYSYEEGAVFLEKLH
KAGIHYIPIVDSALYIPDPNNASDAYDTYTRGAELDVFLKNPDGSTYIGAVWPGYTVFADWHH
PKAGDFWANELVTWHERVAFDGIWIDMNEVSSFCVGSCGSGKLSQNPVHPPFSLPGEPGNIIY
DYPEGFNATNSTEAASASAASASQASAAAATGQSAATTTTPYLRTTPTPGVRDVNHPPYVINH
AQTGHDLAVHAVSPNATHSDGVQEYDVHSLYGHSIIRATYEGLLKVFPEKRPFIIGRSTFAGT
GKWAGHWGGDNNSKWSYMFWSIPQALQFSLFGVPMFGVDTCGFNGNTDEELCNRWMQLSAFFP
FYRNHNVLSAISQEPYRWASVAEASKAAMKIRYAILPYMYTLFQQAHTTGSTVMRALAWEFPN
DPSLAAVDTQFLLGPSIMVVPVLAPQATSVKGVFPGIKQGEVWYDWYTQTAVDAQPHVNTTIA
APLGHIPVFVRGDSVLPMQEPALTTRDARNTPWTILAALGDKGTASGELYLDDGESLEPNATL
TVTFKATKSSLSAEPRGNWQEKNALGNVNVLGVAHKPNGVTLNGKAVPAASVHYNSTSQVLSV
TDLQKMTSKGAFANNWVLKW

Additional α-glucosidase include but are not limited to those having at least 70%, at least 80%, at least 90%, at least 95%, at least 97%, or even at least 99% amino acid sequence identity to SEQ ID NO: 1, SEQ ID NO: 2, SEQ ID NO: 3, and/or SEQ ID NO: 4.

In some embodiments, α-glucosidase is added to corn fiber in an amount of less than 100, less than 70, less than 50, less than 40, less than 30, less than 25, or even less than 20 μg enzyme per g fiber. In some embodiments, the increase in glucose release from corn fiber is at least 1%, at least 2%, at least 5%, at least 10%, at least 20%, at least 30%, at least 40%, at least 50%, at least 60%, or even at least 70%, compared to the amount of glucose released under equivalent conditions in the absence of α-glucosidase. In some embodiments, the increase in ethanol obtained from corn fiber is at least 0.1%, at least 0.2%, at least 0.5%, at least 1.0%, or even at least 2.0% compared to the amount of produced under equivalent conditions in the absence of α-glucosidase. The absence of α-glucosidase encompasses conditions where a minor amount of background α-glucosidase may be present, i.e., less than 5, less than 3, less than 2, or even less than 1 μg enzyme per g fiber.

IV. Corn Fiber Materials from Predominantly Corn Kernel Feedstocks

The present compositions and methods involve the addition of α-glucosidase to corn fiber materials derived from a predominantly corn kernel feedstock that has been pretreated in preparation for enzymatic digestion.

In some embodiments, the compositions and methods relate to producing additional ethanol from a stillage by-product of an ethanol production facility that utilizes predominantly corn kernel feedstock. Whole stillage, or simply stillage, refers to the solid mixture that remains after distilling ethanol and other low molecular components from a fermentation mixture (or beer). Typically, whole stillage falls to the bottom of distillation columns and is eventually transferred to one or more holding tanks to make room for a fresh batch of beer. Whole stillage may then be processed, e.g., via centrifugation, to separate it into mostly-liquid thin stillage and wet distillers' grain (or wet cake). Wet cake may be sold as animal feed directly or dried to produce distillers' dry grain (DDG). Syrup obtained from the drying the thin stillage may be added to the DDG to produce DDG with solubles (DDGS), which is a preferred form of animal feed.

The present compositions and methods are directed at using whole stillage for the production of additional fermentable sugars. Derivatives of whole stillage, such as thin stillage, wet cake, DDG, and DDGS may also be used, although the additional processing steps are deemed unnecessary.

An exemplary process for the use of whole stillage for the production of fermentable sugars is described in U.S. Pat. No. 8, 633,033 (Assigned to Quad County Corn Processors, Holstein, Iowa, USA), which is herein incorporated by reference in its entirety. However, the importance and commercial value of using α-glucosidase to release fermentable carbohydrates from pretreated corn fiber in a starch-hydrolysis process are previously undescribed.

In other embodiments, the present compositions and methods relate to producing additional ethanol from corn fiber materials that are mechanically separated from carbohydrate material in a predominantly corn kernel feedstock at the beginning of a corn starch production process. The corn fibers are typically separated mechanically, primarily to remove unwanted fiber from carbohydrate portion of the feedstock, which fibers can lead to fouling and clogging equipment and increase the demand for material handling. Following chemical pretreatment of the fibers to render them amenable to enzymatic digestion, the fibers are subsequently treated with α-glucosidase.

In some embodiments, the amount of fiber present in the corn fiber materials derived from a predominantly corn kernel feedstock is less than about 30%, less than about 25%, less than about 20%, less than about 15%, less than about 12%, or even less than about 10% of the total dry weight of the substrate. Exemplary amounts are about 1-30%, about 1-25%, about 1-20%, about 1-15%, and about 1-12%.

### V. Chemical, Thermal, or Mechanical Tretreatment

The present compositions and methods require some form of chemical, thermal, or mechanical pretreatment, or combination of, the corn fibers to prepare them for enzymatic digestion. Exemplary treatments are with dilute acid, optionally with heat and pressure, or more concentrated acid, typically at lower temperatures and pressures. Alternative treatments are with dilute ammonia. The optimum pH for α-glucosidase treatment largely depend on the optimum pH for the enzyme and is not believed to be critical.

In another embodiment, heating of the fiber substrate is performed wherein high pressure steam is injected into the substrate mixture to increase its temperature to about 215-300° F. (i.e., about 102-150° C.), preferably in a container or vessel able to maintain a pressure above the vapor pressure of water in order to avoid boiling the sample. Heating by steam injection is beneficial because it aids in disruption of the fiber structure to improve subsequent enzymatic hydrolysis.

In another embodiment, mechanical pretreatment is used to produce uniform particle size of less than about 1,600 microns (μm). In some embodiments, at least 95% of the pretreated biomass particles are mechanically treated to have a particle size of from about 100 microns to about 800 microns, or a particle size from about 100 microns to about 500 microns. Such pretreated biomass particles can be generated by, for example, using a hammer mill or a colloid mill.

### EXAMPLES

### Example 1. Cloning of Paecilomyces variotii α-glucosidase (PvaGlu1)

Paecilomyces variotii NRRL1115 strain was selected as a potential source for various enzymes, useful for industrial applications. The entire genome of the P. variotii NRRL1115 strain was sequenced and the nucleotide sequence of a putative α-glucosidase, designated “PvaGlu1” was identified by sequence identity. The gene encoding PvaGlu1 is set forth as SEQ ID NO:5:

ATGGGCGGCTTCACCCACTACATGCTCGCTTCCGCTTGGCTGCCTCTGAC
CCTGGGCGCCGCCATCTCTCCCGCTGCTACCAGCAGCACCGCTTCGTGGG
GCCCCGTTTTCACCGTCCCCGCCTCCGCCGACGAGGGCGCTCAGCTCATT
GCCAACATTAACGACCCCCAGTCTGTTAACGCCCAGACCGTTTGCCCCGG
CTACGTTGCTTCTAACGTCCAGAACAACGAGTTCGGCTTCACCGCCACCC
TGAACCTCGCCGGCAAGGCTTGCAACGTTTACGGCACCGACGTCGATAGC
CTGAACCTGACCGTCCAGTACCAGGCGAGTGACCGACTGAACATTAACAT
TGGCCCCGCCCACGTTGACGCCTCCAACGAGTCTTGGTACATTCTGAGCG
ACGACCTGGTTTACAAGCCTACCGTGGACGGCACCGCTTCTATCTCTCAG
TCTGACCTGCTGGTTAGCTGGTCTAACGAGCCTTCTTTCAACTTCAAGGT
TATTCGTAAGGCTAACAAGGACGTCCTGTTCAACACCGAGGGCACCGTCC
TCGTTTACGAGAACCAGTTCATCGAGTTCGTCAGCGCTCTCCCTGAGAAC
TACAACCTGTACGGCCTGGGCGAGCGCATTCACGGCCTGCGACTCGGCAA
CAACTTCACCGCCACCACCTACGCCGCCGACGCCGCCGACCCCATCGACG
CTAACATATATGGCACCCACCCTTTCTACCTGGACACCCGATACTACGAG
GTTGACTCCAAGCAGGGCACCTACACCCTGCTGACCACCAACGAGACCGA
CCAGTCTAAGGAATACACCTCTTTCTCTCACGGCGTTTTCCTCCGAAACG
CTCACGGCCAGGAAGTTCTGCTGCGCCCTGAGGGCATCACCTGGCGAACC
CTGGGCGGCAGCATTGACCTGTACTTCTACTCCGGCCCTACCCAGGCTGA
CGTCACCCGATCTTACCAGACCAGCACCGTTGGCCTGCCTACCATGCAGC
AGTACTACACCTTCGGCTACCACCAGTGCCGATGGGGCTACCAGAACTGG
TCTGTCATGGCTGACGTTGTTAGCTCTTTCGCCAAGTTCCAGATTCCTCT
GGAGACCATTTGGAGCGACATCGACTACATGAACGCTTACCGAGACTTCG
AGAACGACCCTATCCGATTCTCTTACTCTGAGGGCGCTGAGTTCCTCGGC
CAGCTGCACGAGAACGGCCAGCACTGGGTTCCTATTGTTGACTCCGCCAT
CTACATCCCTAACAGCGAGAACGCCAGCGACGCTTACGACGTTTACACCC
GAGGCGAGGCCGACGGCGTTTGGATGACCAACCCCGACGGCAGCCTGTAC
ATTGGCGCCGTTTGGCCCGGCTACACCGTTTTCCCCGACTGGCACAACCC
TAAGGCTCACGAGTTCTGGTCTAACGAGATTGCTACCTGGCACCAGAAGG
TCGCTTTCGACGGCATTTGGATTGACATGTCTGAGGTCAGCTCTTTCTGC
GTTGGCTCTTGCGGCACCGGCAACCTGACCCTCAACCCCGTCCACCCTTC
TTTCCTGCTCCCCGGCGAGCCCGGCGCTGTTATATATGACTACCCTGAGT
CTTTCAACGTCACCAACTCTACCGAGGCTGCTTCTGCTTCAGCCGCATCG
GTCTCCCAGGCTGCTGCTACCGCTTCTGCCTCTGCCTCAACCACCACCTC
TTACCTGCGAACCACCCCTACCCCCGGCGTCCGAGACGTTAACCACCCTC
CTTACGTTATTAACAACGTCCAGCCTGGCCACGACCTCGCCGTCCACGCC
GTTAGCCCCAACGCTACCCACATTGACGGCGTTAGCGAGTACGACGTCCA
CAACCTGTGGGGCTACCAGATTCTGAACGCTACCTACCACGGCCTCCTCA
AGGTTTGGGAGGACAAGCGCCCTTTCATTATTGGCCGAAGCACCTTCGCT
GGCAGCGGCAAGTGGGCTGGCCACTGGGGCGGCGACAACACCTCTCTGTG
GGCTTACATGTTCTTCAGCATTCCTCAGGCTCTGTCTTTCAGCCTGTTCG
GCATCCCCATGTTCGGCGTTGACACCTGCGGCTTCAACGGCAACTCCGAC
GAAGAACTGTGCAACCGATGGATGCAGCTGTCTGCTTTCTTCCCTTTCTA
CCGAAACCACAACGTCCTCAGCGCTATTAGCCAAGAACCTTACGTTTGGG
CCTCCGTCATTGACGCTTCTAAGGCCGCCATGAAGATTCGATACGCTCTG
CTGCCTTACATATACACCCTGTTCTACCTCGCTCACACCACCGGCAGCAC
CGTCATGCGAGCTGTCTCTTGGGAGTTCCCTAACGACCCTAGCCTCGCCG
CCATTGACACCCAGTTCCTCCTGGGCCCTAGCCTCATGGTTGTCCCCGTC
CTGGAGCCTCAGGTTGACTACGTTAAGGGCGTTTTCCCCGGCGTTGGCAA
CGGCGAGGTTTGGTACGACTGGTACACCCAGTCTGTTTTCGACGCCAAGC
CCGGCGTTAACACCACCATCAGCGCTCCTCTCGGCCACATCCCCGTTTTC
GTCCGAGGCGGCAGCATTCTGCCTATGCAAGAGCCCGCTCTGACCACCCG
CGACGCTCGAAAGACCCCTTGGGCTCTCCTGACCGCTCTGGGCGGCAACG
GCACCGCTTCTGGCCAGCTGTACATCGACGACGGCGAGTCTATTACCCCT
AACGCCACCCTGAACGTGGACTTCGTTGCTTCCAACTCTAACCTCGTTGC
GTCACCTCGGGGCTCTTGGGTTGAGAAGAACCCTCTCGCTAACGTTACCG
TCCTCGGCGTCCCTACCGCTCCTAGCTCCGTTACCTTCAACGGCGCCGCT
GTTCCTCGCGCTTCCGTCGCTTACAACTCTACCTCCAAGACCCTGTTCGT
TGGCGGCCTGCAAGACTTCACCAAGACCGGCGCTTGGGCTGACAAGTGGG
TTCTCAAGTGG

The amino acid sequence of the PvaGlu1 precursor protein is set forth as SEQ ID NO: 6. The predicted native signal peptide is shown in italics and underlined:

MGGFTHYMLASAWLPLTLGAAISPAATSSTASWGPVFTVPASADEGAQLT
ANINDPQSVNAQTVCPGYVASNVQNNEFGFTATLNLAGKACNVYGTDVDS
LNLTVQYQASDRLNINIGPAHVDASNESWYILSDDLVYKPTVDGTASISQ
SDLLVSWSNEPSFNFKVIRKANKDVLFNTEGTVLVYENQFIEFVSALPEN
YNLYGLGERIHGLRLGNNFTATTYAADAADPIDANTYGTHPFYLDTRYYE
VDSKQGTYTLLTTNETDQSKEYTSFSHGVFLRNAHGQEVLLRPEGITWRT
LGGSIDLYFYSGPTQADVTRSYQTSTVGLPTMQQYYTFGYHQCRWGYQNW
SVMADVVSSFAKFQIPLETIWSDIDYMNAYRDFENDPIRFSYSEGAEFLG
QLHENGQHWVPIVDSAIYIPNSENASDAYDVYTRGEADGVWMTNPDGSLY
IGAVWPGYTVFPDWHNPKAHEFWSNEIATWHQKVAFDGIWIDMSEVSSFC
VGSCGTGNLTLNPVHPSFLLPGEPGAVIYDYPESFNVTNSTEAASASAAS
VSQAAATASASASTTTSYLRTTPTPGVRDVNHPPYVINNVQPGHDLAVHA
VSPNATHIDGVSEYDVHNLWGYQILNATYHGLLKVWEDKRPFIIGRSTFA
GSGKWAGHWGGDNTSLWAYMFFSIPQALSFSLFGIPMFGVDTCGFNGNSD
EELCNRWMQLSAFFPFYRNHNVLSAISQEPYVWASVIDASKAAMKIRYAL
LPYIYTLFYLAHTTGSTVMRAVSWEFPNDPSLAAIDTQFLLGPSLMVVPV
LEPQVDYVKGVFPGVGNGEVWYDWYTQSVFDAKPGVNTTISAPLGHIPVF
VRGGSILPMQEPALTTRDARKTPWALLTALGGNGTASGQLYIDDGESITP
NATLNVDFVASNSNLVASPRGSWVEKNPLANVTVLGVPTAPSSVTFNGAA
VPRASVAYNSTSKTLFVGGLQDFTKTGAWADKWVLKW

The amino acid sequence of the mature form of PvaGlu1 (also referred to herein as E3 GH31 α-glucosidase) is set forth as SEQ ID NO:3, which is shown, above.

### Example 2. Expression and Purification of PvaGlu1

The nucleotide sequence of a synthetic gene encoding PvaGlu1 optimized for expression in Trichoderma reesei is set forth as SEQ ID NO. 7:

ATGGGCGGCTTCACCCACTACATGCTCGCTTCCGCTTGGCTGCCTCTGAC
CCTGGGCGCCGCCATCTCTCCCGCTGCTACCAGCAGCACCGCTTCGTGGG
GCCCCGTTTTCACCGTCCCCGCCTCCGCCGACGAGGGCGCTCAGCTCATT
GCCAACATTAACGACCCCCAGTCTGTTAACGCCCAGACCGTTTGCCCCGG
CTACGTTGCTTCTAACGTCCAGAACAACGAGTTCGGCTTCACCGCCACCC
TGAACCTCGCCGGCAAGGCTTGCAACGTTTACGGCACCGACGTCGATAGC
CTGAACCTGACCGTCCAGTACCAGGCGAGTGACCGACTGAACATTAACAT
TGGCCCCGCCCACGTTGACGCCTCCAACGAGTCTTGGTACATTCTGAGCG
ACGACCTGGTTTACAAGCCTACCGTGGACGGCACCGCTTCTATCTCTCAG
TCTGACCTGCTGGTTAGCTGGTCTAACGAGCCTTCTTTCAACTTCAAGGT
TATTCGTAAGGCTAACAAGGACGTCCTGTTCAACACCGAGGGCACCGTCC
TCGTTTACGAGAACCAGTTCATCGAGTTCGTCAGCGCTCTCCCTGAGAAC
TACAACCTGTACGGCCTGGGCGAGCGCATTCACGGCCTGCGACTCGGCAA
CAACTTCACCGCCACCACCTACGCCGCCGACGCCGCCGACCCCATCGACG
CTAACATATATGGCACCCACCCTTTCTACCTGGACACCCGATACTACGAG
GTTGACTCCAAGCAGGGCACCTACACCCTGCTGACCACCAACGAGACCGA
CCAGTCTAAGGAATACACCTCTTTCTCTCACGGCGTTTTCCTCCGAAACG
CTCACGGCCAGGAAGTTCTGCTGCGCCCTGAGGGCATCACCTGGCGAACC
CTGGGCGGCAGCATTGACCTGTACTTCTACTCCGGCCCTACCCAGGCTGA
CGTCACCCGATCTTACCAGACCAGCACCGTTGGCCTGCCTACCATGCAGC
AGTACTACACCTTCGGCTACCACCAGTGCCGATGGGGCTACCAGAACTGG
TCTGTCATGGCTGACGTTGTTAGCTCTTTCGCCAAGTTCCAGATTCCTCT
GGAGACCATTTGGAGCGACATCGACTACATGAACGCTTACCGAGACTTCG
AGAACGACCCTATCCGATTCTCTTACTCTGAGGGCGCTGAGTTCCTCGGC
CAGCTGCACGAGAACGGCCAGCACTGGGTTCCTATTGTTGACTCCGCCAT
CTACATCCCTAACAGCGAGAACGCCAGCGACGCTTACGACGTTTACACCC
GAGGCGAGGCCGACGGCGTTTGGATGACCAACCCCGACGGCAGCCTGTAC
ATTGGCGCCGTTTGGCCCGGCTACACCGTTTTCCCCGACTGGCACAACCC
TAAGGCTCACGAGTTCTGGTCTAACGAGATTGCTACCTGGCACCAGAAGG
TCGCTTTCGACGGCATTTGGATTGACATGTCTGAGGTCAGCTCTTTCTGC
GTTGGCTCTTGCGGCACCGGCAACCTGACCCTCAACCCCGTCCACCCTTC
TTTCCTGCTCCCCGGCGAGCCCGGCGCTGTTATATATGACTACCCTGAGT
CTTTCAACGTCACCAACTCTACCGAGGCTGCTTCTGCTTCAGCCGCATCG
GTCTCCCAGGCTGCTGCTACCGCTTCTGCCTCTGCCTCAACCACCACCTC
TTACCTGCGAACCACCCCTACCCCCGGCGTCCGAGACGTTAACCACCCTC
CTTACGTTATTAACAACGTCCAGCCTGGCCACGACCTCGCCGTCCACGCC
GTTAGCCCCAACGCTACCCACATTGACGGCGTTAGCGAGTACGACGTCCA
CAACCTGTGGGGCTACCAGATTCTGAACGCTACCTACCACGGCCTCCTCA
AGGTTTGGGAGGACAAGCGCCCTTTCATTATTGGCCGAAGCACCTTCGCT
GGCAGCGGCAAGTGGGCTGGCCACTGGGGCGGCGACAACACCTCTCTGTG
GGCTTACATGTTCTTCAGCATTCCTCAGGCTCTGTCTTTCAGCCTGTTCG
GCATCCCCATGTTCGGCGTTGACACCTGCGGCTTCAACGGCAACTCCGAC
GAAGAACTGTGCAACCGATGGATGCAGCTGTCTGCTTTCTTCCCTTTCTA
CCGAAACCACAACGTCCTCAGCGCTATTAGCCAAGAACCTTACGTTTGGG
CCTCCGTCATTGACGCTTCTAAGGCCGCCATGAAGATTCGATACGCTCTG
CTGCCTTACATATACACCCTGTTCTACCTCGCTCACACCACCGGCAGCAC
CGTCATGCGAGCTGTCTCTTGGGAGTTCCCTAACGACCCTAGCCTCGCCG
CCATTGACACCCAGTTCCTCCTGGGCCCTAGCCTCATGGTTGTCCCCGTC
CTGGAGCCTCAGGTTGACTACGTTAAGGGCGTTTTCCCCGGCGTTGGCAA
CGGCGAGGTTTGGTACGACTGGTACACCCAGTCTGTTTTCGACGCCAAGC
CCGGCGTTAACACCACCATCAGCGCTCCTCTCGGCCACATCCCCGTTTTC
GTCCGAGGCGGCAGCATTCTGCCTATGCAAGAGCCCGCTCTGACCACCCG
CGACGCTCGAAAGACCCCTTGGGCTCTCCTGACCGCTCTGGGCGGCAACG
GCACCGCTTCTGGCCAGCTGTACATCGACGACGGCGAGTCTATTACCCCT
AACGCCACCCTGAACGTGGACTTCGTTGCTTCCAACTCTAACCTCGTTGC
GTCACCTCGGGGCTCTTGGGTTGAGAAGAACCCTCTCGCTAACGTTACCG
TCCTCGGCGTCCCTACCGCTCCTAGCTCCGTTACCTTCAACGGCGCCGCT
GTTCCTCGCGCTTCCGTCGCTTACAACTCTACCTCCAAGACCCTGTTCGT
TGGCGGCCTGCAAGACTTCACCAAGACCGGCGCTTGGGCTGACAAGTGGG
TTCTCAAGTGG

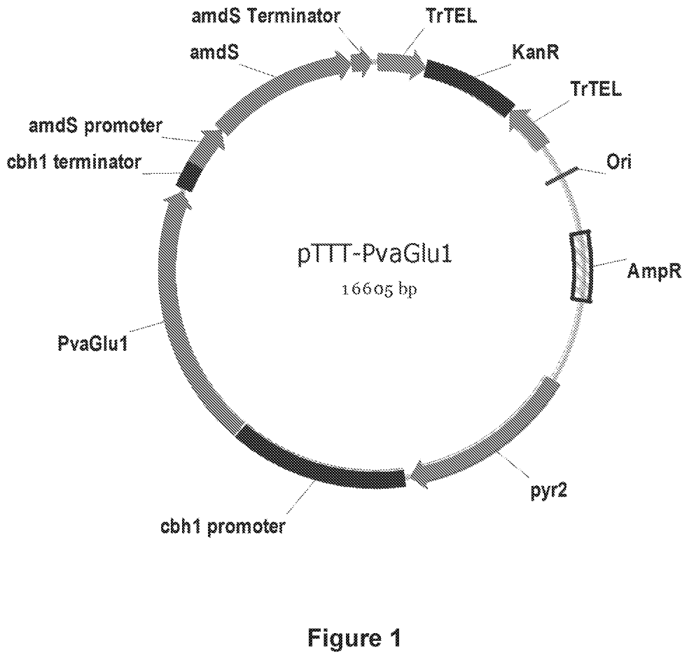

The foregoing gene was inserted into the pTTT expression vector (described in WO2015/017256), resulting in plasmid pTTT-PvaGlu1 (FIG. 1). pTTT-PvaGlu1 was transformed into a quad-deleted Trichoderma reesei strain (described in WO 05/001036) using protoplast transformation (Te'o et al. (2002) J. Microbiol. Methods 51:393-99). Transformants were selected and grown using the methods described in WO 2016/138315. Supernatants from these cultures were used to confirm the protein expression by SDS-PAGE analysis and in assays to measure enzyme activity.

A seed culture of the transformed cells mentioned was subsequently grown in a 2.8 L fermenter in defined medium. Fermentation broth was sampled at elapsed times of 42 hours, 65 hours, and 95 hours for SDS-PAGE analysis and measurements of dry cell weight, residual glucose and extracellular protein concentration. Following centrifugation, filtration and concentration, 500 mL of concentrated sample having a protein concentration of 10.7 g/L was obtained. PvaGlu1 was purified via hydrophobic interaction chromatography (HIC) followed by ammonium sulfate precipitation, and then stored at −20° C. until needed.

### Example 3. Substrate specificity of PvaGlu1

Substrate specificity of PvaGlu1 was assayed based on the release of glucose following incubation with isomaltose, maltose, trehalose, panose, maltulose, sucrose, leucrose, nigerose, kojibiose, maltotriose or maltoheptaose. The rate of glucose release was measured using a coupled glucose oxidase/peroxidase (GOX/HRP) method (Ngo, T. T. and Lenhoff, H. M. (1980) Anal. Biochem. 105:389-397). Glucose was quantified as the rate of oxidation of 2,2′-azino-bis 3-ethylbenzothiazoline-6-sulfonic acid (ABTS) by peroxide which was generated from coupled GOX/HRP enzymes reacted with glucose.

Substrate solutions were prepared by mixing 9 mL of each substrate mentioned above (1% in water, w/v), 1 mL of 0.5 M pH 5.0 sodium acetate buffer, and 40 μL of 0.5 M calcium chloride in a 15-mL conical tube. Coupled enzyme (GOX/HRP) solution with ABTS was prepared in 50 mM sodium acetate buffer (pH 5.0), with the final concentrations of 2.74 mg/mL ABTS, 0.1 U/mL HRP, and 1 U/mL GOX.

Serial dilutions of α-glucosidase samples and glucose standard were prepared in Milli Q water. Each α-glucosidase sample (10 μL) was transferred into a new microtiter plate (Corning 3641) containing 90 μL of substrate solution preincubated at 50° C. for 5 min at 600 rpm. The reactions were carried out at 50° C. for 10 min (for isomaltose, maltose, panose, maltulose, nigerose, and kojibiose) and for 60 min (for sucrose and leucrose) with shaking (600 rpm) in a thermomixer (Eppendorf). 10 μL of reaction mixtures as well as 10 μL of serial dilutions of glucose standard were quickly transferred to new microtiter plates (Corning 3641), followed by the addition of 90 μL of ABTS/GOX/HRP solution. The microtiter plates containing the reaction mixture were immediately measured at 405 nm at 11 seconds intervals for 5 min on SoftMax Pro plate reader (Molecular Device). The output was the reaction rate, Vo, for each enzyme concentration. Linear regression was used to determine the slope of the plot Vo versus enzyme dose. The specific activity of α-glucosidase was calculated based on the glucose standard curve using the following equation:

Specific Activity (Unit/mg)=Slope (enzyme)/slope (std)×1000 (1),
where 1 Unit=1 μmol glucose/min.

Except for isomaltose and maltose, the value of the reaction rate with enzyme dosage at 5 ppm was directly used to indicate the enzyme activity on other substrates listed. Using the method mentioned above, substrate specificity of PvaGlu1 was determined and compared with the benchmarks, oligo-1,6-glucosidase (a product purchased from Megazyme) and purified TrTG (transglucosidase from Trichoderma reesei) as shown in Table 1. PvaGlu1 showed broader substrate specificity than the other enzymes, with high activity towards isomaltose, maltose, nigerose, and kojibiose and moderate activity towards panose, maltotriose and maltoheptaose.

TABLE 1
Substrate specificity of purified PvaGlu1 compared with
Oligo-1,6-glucosidase and TrTG.

| | Isomaltose | Maltose | Trehalose | Leucrose | Sucrose | Maltulose | Panose | Maltotriose | Maltoheptaose | Kojibiose | Nigerose |
|---|---|---|---|---|---|---|---|---|---|---|---|
| Enzyme | (U/mg) | (U/mg) | (5 ppm) | (5 ppm) | (5 ppm) | (5 ppm) | (5 ppm) | (5 ppm) | (5 ppm) | (5 ppm) | (5 ppm) |
| oligo-1,6- | 118.2 | 0.0 | 3.3 | 0.8 | 1.2 | 7.1 | 20.3 | 2.2 | 1.2 | 5.1 | 27.3 |
| gluc. | | | | | | | | | | | |
| TrTG | 203.1 | 267.9 | 6.5 | 12.7 | 0.4 | 2.1 | 72.7 | 41.5 | 38.2 | 58.9 | 118.4 |
| PvaGlu1 | 398.2 | 230.8 | 2.4 | 16.9 | 0.7 | 4.2 | 64.9 | 42.4 | 64.3 | 135.1 | 188.6 |

### Example 4. pH profile of PvaGlu1

The effect of pH (from 3.0 to 10.0) on α-glucosidase activity of PvaGlu1 was monitored using isomaltose as a substrate. Buffer working solutions consisted of the combination of glycine/sodium acetate/HEPES (250 mM), with pH varying from 3.0 to 10.0. Substrate solutions were prepared by mixing isomaltose (1% in water, w/v) with 250 mM buffer solution at a ratio of 9:1, containing calcium chloride at a final concentration of 2 mM. Enzyme working solutions were prepared in water at a certain dose (showing signal within linear range as per dose response curve). All the incubations were carried out at 50° C. for 10 min following the same protocol as described in Example 3 for activity of α-glucosidase towards isomaltose. Enzyme activity at each pH was reported as relative activity compared to enzyme activity at optimum pH. The results are shown in Table 2. PvaGlu1 was found to have an optimum pH at 5.0 and retain greater than 70% of maximum activity between 3.4 and 6.6.

TABLE 2
pH profile for PvaGlu1.

| pH | Relative activity (%) |
|---|---|
| 3 | 55 |
| 4 | 92 |
| 5 | 100 |
| 6 | 90 |
| 7 | 53 |
| 8 | 13 |
| 9 | 2 |
| 10 | 0 |

### Example 5. Temperature Profile of PvaGlu1

The effect of temperature (from 40° C. to 85° C.) on α-glucosidase activity of PvaGlu1 was monitored using isomaltose as a substrate. Substrate solutions were prepared by mixing 9 mL of isomaltose (1% in water, w/v), 1 mL of 0.5 M pH 5.0 sodium acetate buffer, and 40 μL of 0.5 M calcium chloride in a 15-mL conical tube. Enzyme working solutions were prepared in water at a certain dose (showing signal within linear range as per dose response curve). Incubations were performed at temperatures from 40° C. to 85° C. for 10 min following the same protocol as described in Example 3 for activity of α-glucosidase towards isomaltose. Activity at each temperature was reported as relative activity compared to enzyme activity at optimum temperature. The results are shown in Table 3. PvaGlu1 was found to have an optimum temperature at 55-60° C. and was able to keep higher than 70% of maximum activity between 49° C. and 68° C.

TABLE 3
Temperature profile of PvaGlu1

| Temp. (° C.) | Relative activity (%) |
|---|---|
| 40 | 43 |
| 44.7 | 63 |
| 49.4 | 73 |
| 55 | 100 |
| 59.7 | 100 |
| 65 | 95 |
| 69.2 | 60 |
| 74.6 | 22 |
| 80 | 20 |
| 85 | 16 |

### Example 6. Glucose Release from Corn Fiber in the Presence of α-glucosidase

Whole stillage, the by-product of a starch ethanol fermentation and distillation, was prepared for a secondary saccharification by treatment with acid (see, e.g., U.S. Pat. No. 8,633,003) and addition of enzymes. Specifically, about 0.4 grams of material containing approximately 20% dry solids was placed in a reaction vessel along with the commercial enzymes ACCELERASE TRIO™ and STARGEN™ 002 (DuPont Industrial Biosciences).

ACCELERASE TRIO™ includes several enzymatic activities, including 2000-2600 CMC U/g endoglucanase activity, >3000 ABX U/g xylanase activity and >2000 pNPG U/g β-glucosidase activity. One carboxy methyl cellulose (CMC) unit is defined as the amount of activity required to liberate 1 μmol of reducing sugars (expressed as glucose equivalents) in one minute at 50° C. (122° F.) at pH 4.8. One ABX unit is defined as the amount of enzyme required to generate 1 μmol of xylose reducing sugar equivalents per minute at 50° C. (122° F.) and pH 5.3. One pNPG unit denotes 1 μmol of nitro-phenol liberated from paranitrophenyl-B-D-glucopyranoside per minute at 50° C. (122° F.) and pH 4.8. ACCELERASE TRIO™ has negligible, if any, α-glucosidase activity. ACCELERASE TRIO™ was dosed at 0.07% wt/vol relative to stillage.

STARGEN™ 002 enzyme contains the α-amylase from Aspergillus kawachi and the glucoamylase from Trichoderma reesei. One glucoamylase Unit (GAU) is the amount of enzyme that will liberate one gram of reducing sugars calculated as glucose per hour from soluble starch substrate under the conditions of the assay. STARGEN™ 002 has minimal α-glucosidase activity, which may be attributable to the small amount α-glucosidase activity associated with α-amylases. STARGEN™ 002 was dosed at 0.017 GAU/gram dry solid stillage.

To the above reactions several different concentrations of purified α-glucosidase was added and the reaction vessels placed in an incubator at 50° C. for 18-24 hr with shaking, followed by termination by the addition of 0.2 M H₂SO₄. Following centrifugation, the supernatant was collected and analyzed by HPLC to measure the concentration of glucose. The name, Carbohydrate Active Enzyme (CAZy) classification, and SEQ ID NO of the enzymes tested is shown in Table 4, below. Note that E3 is PvaGlu1 described in Examples 1-5.

TABLE 4
α-glucosidases tested

| Name | CAZy classification | SEQ ID NO |
|---|---|---|
| E1 | GH31 | 1 |
| E2 | GH31 | 2 |
| E3 | GH31 | 3 |
| E4 | GH31 | 4 |
| D1 | GH97 | n/a |
| D2 | GH97 | n/a |
| D3 | GH97 | n/a |

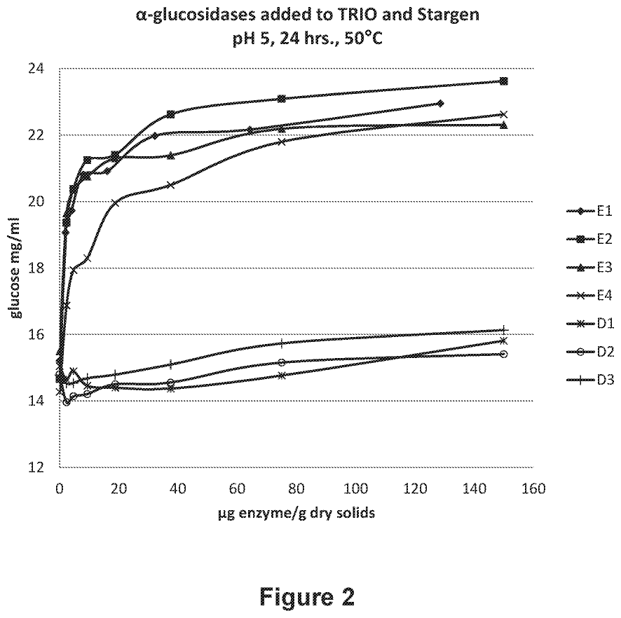

The results are shown in FIG. 2. The addition of both GH31 and GH97 α-glucosidases results in increased glucose release in a dose-dependent manner. However, while the increase observed with GH97 enzymes is only about 1%, the increase observed with GH31 enzymes is as much as 60-70%, with a significant amount of the increase being observed at below 50, and even below 25 μg enzyme per g dry solids.

What is claimed is:

1. A method for increasing the production of fermentable sugars in an ethanol fermentation process, comprising: chemically-pretreating corn fiber material derived from a predominantly corn kernel feedstock; and contacting the pretreated corn fiber with α-glucosidase to release fermentable sugars from the corn fiber; wherein the contacting with α-glucosidase releases fermentable sugars from the corn fiber material derived from a predominantly corn kernel feedstock that otherwise would not be available for fermentation.

2. The method of claim 1, wherein the corn fiber product is the stillage resulting from distillation of a fermentation product, or a derivative, thereof.

3. The method of claim 1, wherein the corn fiber product is produced by physically separating corn fiber from starch in a substrate for ethanol fermentation.

4. The method of claim 1, wherein the corn fiber product is exposed to a temperature of at least 80° C. prior to the contacting with α-glucosidase.

5. The method of claim 1, wherein the corn fiber product is subjected to an alcohol distillation step prior to the contacting with α-glucosidase.

6. The method of claim 1, wherein the corn fiber product contains between about 1-12% corn fiber (wt/wt).

7. The method of any of the preceding claims, wherein the pretreatment is acid pretreatment.

8. The method of any of the preceding claims, wherein the pretreatment is alkaline pretreatment.

9. The method of any of the preceding claims, wherein the α-glucosidase is a GH31 enzyme.

10. The method of any of the preceding claims, wherein the α-glucosidase has at least 70% amino acid sequence identity to a polypeptide having an amino acid sequence selected from the group consisting of SEQ ID NO: 1, SEQ ID NO: 2, SEQ ID NO: 3 and SEQ ID NO: 4.

11. The method of any of the preceding claims, wherein the contacting with α-glucosidase is performed in the presence of one or more additional enzymes.

12. A method for increasing the production of fermentable sugars from chemically-pretreated stillage, comprising:
fermenting a predominantly corn kernel feedstock with an ethanolagen to produce ethanol;
evaporating at least a portion of the ethanol and recovering stillage;
chemically-pretreating the stillage;
contacting the stillage with α-glucosidase to release fermentable sugars;
fermenting the released fermentable sugars with an ethanolagen to produce additional ethanol; and
recovering the additional ethanol;
wherein the contacting with α-glucosidase releases fermentable sugars from the corn fiber material derived from the predominantly corn kernel feedstock that otherwise would not be available for fermentation.

13. The method of claim 12, wherein the pretreatment is acid pretreatment.

14. The method of claim 12, wherein the pretreatment is alkaline pretreatment.

15. The method of any of claims 12-14, wherein the corn fiber product contains between about 1-12% corn fiber (wt/wt).

16. The method of any of claims 12-15, wherein the α-glucosidase is a GH31 enzyme.

17. The method of any of claims 12-16, wherein the α-glucosidase has at least 70% amino acid sequence identity to a polypeptide having an amino acid sequence selected from the group consisting of SEQ ID NO: 1, SEQ ID NO: 2, SEQ ID NO: 3 and SEQ ID NO: 4.

18. The method of any of claims 12-17, wherein the contacting with α-glucosidase is performed in the presence of one or more additional enzymes.

19. A polypeptide having α-glucosidase activity, selected from the group consisting of:
(a) a polypeptide comprising an amino acid sequence having preferably at least 94% identity to the polypeptide of SEQ ID NO: 3;
(b) a polypeptide encoded by a polynucleotide that hybridizes under preferably at least low stringency conditions, more preferably at least medium stringency conditions, even more preferably at least medium-high stringency conditions, most preferably at least high stringency, and even most preferably at least very high stringency conditions with
(i) the mature polypeptide coding sequence of SEQ ID NO: 7,
(ii) the genomic DNA sequence comprising the mature polypeptide coding sequence of SEQ ID NO: 7, or
(iii) a full-length complementary strand of (i) or (ii);
(c) a polypeptide encoded by a polynucleotide comprising a nucleotide sequence having preferably at least 94% identity to the polypeptide coding sequence of SEQ ID NO: 3;
(d) a variant comprising a substitution, deletion, and/or insertion of one or more (e.g., several) amino acids of the polypeptide of SEQ ID NO: 3;
(e) a mature polypeptide produced by the processing of the polypeptide of SEQ ID NO: 6 by a signal peptidase or post translational modification during secretion from an expression host; and
(f) a fragment of a polypeptide of (a), (b), (c), (d), or (e) that has α-glucosidase activity.

20. A polynucleotide comprising a nucleotide sequence that encodes the polypeptide of claim 19.
